(19) United States
(12) Patent Application Publication
(10) Pub. No.: US 2008/0153098 A1
(43) Pub. Date: Jun. 26, 2008

(54) Methods for diagnosing and treating breast cancer based on a HER/ER ratio

(76) Inventors: David L. Rimm, Branford, CT (US); Gino Pereira, Oxford, CT (US); Malini Harigopal, New Haven, CT (US); Jennifer Gitnane, New Haven, CT (US); Marisa P. Dolled-Filhart, New Haven, CT (US); Robert L. Camp, Stamford, CT (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/953,611

(22) Filed: Dec. 10, 2007

Related U.S. Application Data
(63) Continuation of application No. PCT/US06/22819, filed on Jun. 9, 2006.
(60) Provisional application No. 60/689,149, filed on Jun. 9, 2005.
(60) Provisional application No. 60/731,427, filed on Oct. 28, 2005.

(51) Int. Cl. C12Q 1/68

(52) U.S. Cl. 435/6

(57) ABSTRACT

Provided are improved, quantitative methods for determining whether a subject is likely to respond to a breast cancer therapy, methods for selecting breast cancer therapies and methods for diagnosis and prognosis of breast cancer in subjects.

### CROSS-REFERENCE TO RELATED APPLICATIONS

This application is a continuation of International Application No. PCT/US06/022819, filed Jun. 9, 2006, which claims priority to U.S. Provisional Application Nos. 60/689,149, filed on Jun. 9, 2005, and 60/731,427, filed on Oct. 28, 2005. The contents of each of these applications is expressly incorporated herein by reference in their entirety.

### BACKGROUND OF THE INVENTION

Breast cancer is the third most common cancer, and the most common cancer in women, as well as a cause of disability, psychological trauma, and economic loss. Breast cancer is the second most common cause of cancer death in women in the United States, in particular for women between the ages of 15 and 54.

Despite recent advances, one challenge of cancer treatment remains to target specific treatment regimens to pathogenically distinct tumor types, and ultimately personalize tumor treatment in order to maximize outcome. Hence, a need exists for tests that simultaneously provide predictive information about patient responses to the variety of treatment options. This is particularly true for breast cancer, the biology of which is poorly understood. It is clear that the classification of breast cancer into a few subgroups, such as ErbB2+ subgroup, and subgroups characterized by low to absent gene expression of the estrogen receptor (ER) and a few additional transcriptional factors (Perou et al., Nature 406:747-752 (2000)) does not reflect the cellular and molecular heterogeneity of breast cancer, and does not allow the design of treatment strategies maximizing patient response.

A multitude of breast cancer mRNA profiling studies has stratified breast cancer and defined gene sets which correlate with outcome. These studies have resulted in plans for prospective application of nucleic acid based tests to select patients that do not need further therapy after their primary resection. However, the number of genes used to predict patient outcome or define tumor subtypes by RNA expression studies is variable, non-overlapping, and generally requires specialized technologies that are beyond those used in the routine pathology lab. Immuno-histochemical (IHC) studies can be done with many fewer markers, but suffer from the inherent flaw of subjective analysis and variable reproducibility.

The current Herceptest™ or the FISH based test for HER2 amplification are the standard companion diagnostics for Herceptin™. However, even when either or both of these tests are “positive,” only around 50% of these patients will respond to therapy.

Given the cardiotoxicity and expense of this therapy, it would be advantageous to be able to more exactly identify subject that will respond positively to therapy.

### SUMMARY OF THE INVENTION

Provided herein are methods of selecting and evaluating therapies for breast cancer that comprise quantitatively evaluating the ratio of expression of a HER family member to the level of expression of ER in biological samples, particularly tissue samples. Because a ratio is measured, the methods allow internal standardization and normalization and can reveal biologically significant relationships that may be obscured by biological sample preparation. Evaluation of the ratio of expression of a HER family member to the level of expression of ER in biological samples may also comprise methods of diagnosing, staging and prognosing breast cancer.

Further provided are quantitative multiplex assays for selecting and evaluating therapies for breast cancer. The ability to multiplex markers allows for greater complexity in the assessment of multiple biomarkers that can contribute to predicting patient outcome. The quantitative multiplex assays may also comprise methods of diagnosing, staging and prognosing breast cancer. The HER/ER ratio assay may be performed in conjunction with any of the multiplex assays.

Compositions and kits for the practice of the methods are also described herein. These embodiments of the present invention, other embodiments, and their features and characteristics will be apparent from the description, drawings, and claims that follow.

### BRIEF DESCRIPTION OF THE DRAWINGS

FIG. 1 depicts the effect of quantitative analysis and multiplexing HER molecules on the prognostic value. (A) shows the KM curve of traditional scoring of HER2 staining in a cohort of 550 patients (the distribution of scores is inset). (B) shows analysis of the same set using AQUA™ based scoring. (C) shows the results of cluster-based analysis.

FIG. 2 depicts a table of antibody staining conditions, sources and references of microarray studies.

FIG. 3 depicts a table with the results of Cox univariate analysis of breast cancer samples with continuous variables on 250-case cohort (5-year survival disease free analysis).

FIG. 4 depicts immunofluorescent staining of breast cancer tissue microarray tumors. Images of tissue microarray breast tumor cores are shown at 40× magnification, with the marker staining shown in red, and cytokeratin staining (green) and DAPI staining (blue) in the inset to indicate the presence of tumor in that area in which we show marker expression. Estrogen receptor staining is shown in (A) as an example of high expression, and low expression in (D). High and low expression of NAT1 is shown in (B) and (E), and GATA3 high and low expression is shown in (C) and (F), respectively.

FIG. 5 depicts unsupervised hierarchical clustering of thirty-five estrogen receptor and related markers. The heat maps shown have the overall similarity ordering of the breast cancer tumors on the vertical axis, and the ordering of antibody immunoreactivities (AQUA™ scores normalized by z-score transformation as described in the methods section) on the horizontal axis. Black indicates protein expression level equal to the mean, green indicates protein expression levels below the mean, red indicates protein expression level above the mean. The intensity of the color represents the magnitude of expression, with brightest red or green representing the higher or lower respectively, expression compared to the mean. The branch lengths and pattern demonstrate the relatedness of the tumors on the vertical axis and the antibody staining on the horizontal axis. (A) Heat map of 161 tumors (with 80% filtering) for thirty-five markers related to estrogen responsiveness and estrogen receptor status. Gray indicates missing values. The yellow highlighted section indicates the markers that clustered in a small group with estrogen receptor (GATA3 and NAT1). (B) Kaplan-Meier survival curve analysis of the Clusters A, B, C and D as designated in (A).

FIG. 6 depicts a table with the results of Cox univariate analysis of breast cancer samples with nominalized variables for the entire 675-case breast cancer cohort.

FIG. 7 depicts unsupervised hierarchical clustering of NAT1, GATA3 and ER. (A) Heat map of 502 tumors with values for NAT1, GATA3 and ER protein expression. (B) Kaplan-Meier survival curve analysis of the two major cluster groups (Cluster 1, n=210, and Cluster 2, n=292) as shown in (C) at 5-year disease-specific survival. (C) This Kaplan-Meier curve separates out clusters 1A (n=84), 1B (n=34), 1C (n=92) and B (n=292) including all patients for which we had censor and survival data (n=446).

FIG. 8 depicts a table with the results of Cox univariate analysis of breast cancer samples with nominalized variables for 250-case tissue microarray (5-year survival).

FIG. 9 depicts Kaplan-Meier survival curve analysis of low, intermediate and high risk patient groups. The scores of patients for the training set-generated model equating a single point for each criteria met of: ER>4.6, GATA3>33.8, NAT1>18.6 were stratified into 4 groups of high risk (Group D, 0 points), intermediate risk 1 (Group C, 1 point), intermediate risk 2 (Group B, 2 points) and low risk (Group A, 3 points). (A) Kaplan-Meier survival curves of the training set tumors. (B) Kaplan-Meier survival curves of the validation set tumors. (C) Kaplan-Meier survival curves of the validation set from the Swedish cohort.

FIG. 10 depicts a table with the results of multivariate analysis of multiplex model with clinical factors.

### DETAILED DESCRIPTION OF THE INVENTION

For convenience, before further description of the present invention, certain terms employed in the specification, examples and appended claims are defined here.

The singular forms “a”, “an”, and “the” include plural references unless the context clearly dictates otherwise.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

The term “ER”, an abbreviation for “estrogen receptor,” refers to a known nuclear receptor having a predicted molecular weight of about 66-kDa, that is activated by estrogenic steroid hormones such as estradiol. The active form of the protein enhances expression of genes involved in the formation of secondary sexual characteristics in mammalian females. An estrogen receptor can be an allele, polymorphic variant, interspecies homolog, or any subsequence thereof that exhibits estrogenic steroid hormone-binding activity.

As used herein, “estrogen receptor gene” is a wild-type or mutant nucleotide sequence that encodes an estrogen receptor described herein, and conservatively modified variants thereof. An example of an estrogen receptor gene is described in Greene et al., 1986, Science 231(4742):1150-4. One of ordinary skill in the art will recognize that certain modifications, additions, and deletions may be made to the estrogen receptor gene sequence which will not affect the function or activity of the gene product. Such variants are included within this definition. An example of a mutant estrogen receptor gene is K206A, in which the lysine at position 206 is replaced by alanine.

“Gene” or “recombinant gene” refers to a nucleic acid molecule comprising an open reading frame and including at least one exon and (optionally) an intron sequence. “Intron” refers to a DNA sequence present in a given gene which is spliced out during mRNA maturation.

By “gene product” it is meant a molecule that is produced as a result of transcription of a gene. Gene products include RNA molecules transcribed from a gene, as well as proteins translated from such transcripts, as well as mRNA, pre-mRNA nascent transcript(s), transcript processing intermediates, mature mRNA(s) and degradation products.

The term “HER” or “HER family member” refers to a receptor protein tyrosine kinase which belongs to the ErbB receptor family and includes EGFR, HER1, HER2/Neu, HER3, HER4, ErbB2, ErbB3, and ErbB4 receptors, as well as TEGFR (U.S. Pat. No. 5,708,156) and other members of this family to be identified in the future. HER2 will generally comprise an extracellular domain, which may bind an ErbB ligand; a lipophilic transmembrane domain; a conserved intracellular tyro sine kinase domain; and a carboxyl-terminal signaling domain harboring several tyrosine residues which can be phosphorylated. The HER2 may be a native sequence ErbB receptor or an amino acid sequence variant thereof. Preferably the ErbB receptor is native sequence human ErbB receptor.

The term “HER2-based therapy” refers to therapy that includes the use of agents that either directly inhibit HER2 activity (anti-HER2 antibodies such as trastuzumab (Herceptin) and HER2-specific kinase inhibitors) or indirectly inhibit HER2 activity by inhibiting the activity of other HER2 family members that heterodimerize with HER2. For instance, ErbB2 and EGFR can form ErbB2-EGFR heterodimers to transduce signals. Anti-EGFR antibodies and EGFR kinase inhibitors such as gefitinib (ZD1839, Iressa) can thus be used to indirectly block HER2 activity.

The term “hormone therapy” includes the use of antiestrogens or selective estrogen receptor modulators (SERMs), aromatase inhibitors and other agents that can modulate the production or activity of estrogens.

The “level of expression of a gene” or “gene expression level” or simply “expression level” refers to the level of a gene product encoded by the gene in the cell.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids. “Nucleic acid corresponding to a gene” refers to a nucleic acid that may be used for detecting the gene, e.g., a nucleic acid which is capable of hybridizing specifically to the gene.

“Protein”, “polypeptide” and “peptide” are used interchangeably herein when referring to a gene product, e.g., as may be encoded by a coding sequence.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

A “patient”, “subject” or “host” to be treated by the subject method may mean either a human or non-human animal.

B. Methods of Determining the HER2/ER Ratio and/or the Expression of Breast Cancer Markers

Provided are methods for evaluating breast cancer therapies comprising the determination of the HER2/ER ratio in a cell of a subject having breast cancer.

In one embodiment, a method of determining whether a subject having breast cancer is likely to respond to a breast cancer therapy comprises determining the ratio of HER2 expression to ER expression in a cell of said subject, wherein the determined ratio indicates whether the subject is likely to respond to the breast cancer therapy. The breast cancer therapy may be selected, for example, from the group consisting of: HER2-based therapy and hormone therapy.

In another embodiment, a method of selecting a breast cancer therapy for a subject comprises determining the ratio of HER2 expression to ER expression in a cell of said subject, wherein the determined ratio indicates the appropriate therapy for the subject. In certain embodiments, the breast cancer therapy may be selected from either HER2-based therapy or hormone therapy. The determined ratio may indicate that the subject is likely to respond to HER2-based therapy but not hormone therapy, that the subject is likely to respond to hormone therapy but not HER2-based therapy, to both therapies, or to neither therapy.

Also provided are methods of diagnosing, prognosing, and/or staging breast cancer in a subject. Such methods comprise determining the ratio of HER2 expression to ER expression in a cell of said subject, wherein the determined ratio indicates the presence or stage of breast cancer in a subject, or the prognosis for survival of said patient.

The methods comprising determining the ratio of HER2 expression to ER expression may be practiced in a multiplex format along with other assays, e.g., on a single slide or other reaction vessel. For example, the HER2/ER ratio may be determined along with the expression of epidermal growth factor receptor (EGFR), other HER family members such as HER1, HER 2, HER3 and HER4 (and any combination of such members), and/or the various multiplex assays described below and in the Examples, that allows both tests to be done on a single slide.

In other embodiments, methods for evaluating breast cancer therapies or diagnosing, prognosing, and/or staging breast cancer comprise a multiplex determination of biomarker expression not including the determination of the HER2/ER ratio. For example, the methods may comprise determining the expression of any combination of HER1, HER2, HER3 and HER4, ER and progesterone receptor (PR). In other embodiments, the methods may comprise a multiplex marker assay for separation of prognostic groups based on expression of the set of biomarkers: ER, GATA3 and NAT1.

Biomarkers comprising a set to be used in the various multiplex assays described herein may be selected using a genetic algorithm, as further described in the Exemplification below. The National Center for Biotechnology Information (NCBI) accession numbers are as follows: EGFR (HER1): NM_{—}005228; NP_{—}005219; HER2: NM_{—}001005862; NP_{—}004439; HER3: NM_{—}001005915; NP_{—}001973; HER4: NM_{—}005235; NP_{—}005226; ER: NM_{—}000125; NP_{—}000116; GATA3 NM_{—}001002295; NP_{—}001002295; PR: NM_{—}000926; NP_{—}000917 and NAT1: NM_{—}000662; NP_{—}000653.

The level of expression of the various biomarkers used in the assays may be determined by quantifying the level of expression of the genes encoding the biomarkers in the cell, or it may be determined by quantifying the amounts of the biomarker proteins in the cell.

Methods for quantifying the expression level of biomarker genes and ultimately the activity of biomarker proteins are known in the art. For example, the expression level of a biomarker gene can be determined by reverse transcription-polymerase chain reaction (RT-PCR); dotblot analysis; Northern blot analysis and in situ hybridization. Alternatively, the level of a biomarker can be analyzed using an appropriate antibody. Tumor cells can be obtained using known procedures, such as a needle biopsy (See Kim, C. H. et al. J. Virol. 66:3879-3882 (1992)); Biswas, B. et al. Annals NY Acad. Sci. 590:582-583 (1990)); Biswas, B. et al. J. Clin. Microbiol. 29:2228-2233 (1991).

In certain embodiments, the amounts of biomarkers are determined using antibodies against the biomarkers.

In certain embodiments, the level of expression of the biomarkers is determined by determining the AQUA™ score of HER2 and ER, e.g., by using the AQUA™ automated pathology system.

AQUA™ (for Automated Quantitative Analysis) is a method of analysis of absolute measurement of protein expression in situ. This method allows measurements of protein expression within sub-cellular compartments that results in a number directly proportional to the number of molecules expressed per unit area. For example, to measure nuclear estrogen receptor (ER), the tissue is “masked” using keratin in one channel to normalize the area of tumor and to remove the stromal and other non-tumor material from analysis. Then an image is taken using DAPI to define a nuclear compartment. The pixels within the mask and within the DAPI-defined compartment are defined as nuclear. The intensity of expression of ER is measured using a third channel. The intensity of that subset of pixels divided by the number of pixels (to normalize the area from spot to spot) gives an AQUA™ score. This score is directly proportional to the number of molecules of ER per unit area of tumor, as assessed by a standard curve of cell lines with known levels of ER protein expression. This method, including details of out-of-focus light subtraction imaging methods, is described in detail in a Nature Medicine paper (Camp, R. L., Chung, G. G. & Rimm, D. L. Automated subcellular localization and quantification of protein expression in tissue microarrays. Nat Med 8, 1323-7 (2002)), as well as U.S. Ser. No. 10/062,308, filed Feb. 1, 2002, which published as WO 02/086498 are incorporated by reference herein in their entireties.

Exemplary embodiments of the methods of the invention wherein AQUA™ is used to determine, for example, the amount of HER2 and ER and thus the HER2/ER ratio, or the expression of multiple markers in a multiplex format are described in the Exemplification below.

Methods of quantitatively determining biomarker expression may comprise determining the location of the biomarkers in the cell, as well as the quantity of the biomarkers of the cell. AQUA™ is an example of a method which accomplishes both of these goals.

AQUA™ is essentially performed by incubating a tissue section with an appropriate solution containing a first stain that specifically labels the biomarker and a second stain that specifically labels the cellular compartment, thereby obtaining a tissue section that contains a first stain labeled biomarker and a second stain labeled cellular compartment. A first high resolution image is then obtained of the tissue section at a wavelength appropriate to detect the first stain labeled biomarker, wherein the image comprises multiple pixel locations and each of the multiple pixel locations is analyzed to identify pixel locations having an intensity value indicative of the presence of the first stain. A second high resolution image of the tissue section is then obtained at a wavelength appropriate to detect the second stain labeled cellular compartment, wherein the image comprises multiple pixel locations and each of the multiple pixel locations is analyzed to identify pixel locations having an intensity value indicative of the presence of the second stain. The pixel locations in the first and the second images are then compared, wherein pixel locations that have both an intensity value indicative of the presence of the first stain in the first image and an intensity value indicative of the presence of the second stain in the second image indicate that the biomarker is present in the cellular compartment. Quantitation of HER2 and ER based on summing the intensity and dividing by the number of pixels may be used to determine the HER2/ER ratio.

However, other methods of quantitatively determining biomarker expression may also be used. For example, the location of biomarkers in a cell may be accomplished by sub-cellular fractionation followed by quantitation of the biomarkers, e.g. by ELISA analysis, analysis with a polypeptide array, or other method for quantifying biomarker amounts.

In some embodiments, methods of detecting the level of expression of biomarkers may comprise the use of a microarray. Arrays are often divided into microarrays and macroarrays, where microarrays have a much higher density of individual probe species per area. Microarrays may have as many as 1000 or more different probes in a 1 cm² area. There is no concrete cut-off to demarcate the difference between micro- and macroarrays, and both types of arrays are contemplated for use with the invention.

Microarrays are known in the art and generally consist of a surface to which probes that correspond in sequence to gene products (e.g., cDNAs, mRNAs, oligonucleotides, proteins) are bound at known positions. In one embodiment, the microarray is an array (e.g., a matrix) in which each position represents a discrete binding site for a product encoded by a gene (e.g., a protein or RNA), and in which binding sites are present for products of most or almost all of the genes in the organism's genome.

When fluorescently labeled probes are used, the fluorescence emissions at each site of a transcript array may be detected by scanning confocal laser microscopy. When two fluorophores are used, a separate scan, using the appropriate excitation line, is carried out for each of the two fluorophores used. Fluorescent microarray scanners are commercially available from Affymetrix, Packard BioChip Technologies, BioRobotics and many other suppliers. Signals are recorded, quantitated and analyzed using a variety of computer software.

According to the method of the invention, the relative abundance of a gene product in two cells or cell lines is scored as a perturbation and its magnitude determined (i.e., the abundance is different in the two sources of gene product tested), or as not perturbed (i.e., the relative abundance is the same). As used herein, a difference between the two sources of at least a factor of about 25% (gene product from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

In addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In certain embodiments, the data obtained from such experiments reflects the relative expression of each gene represented in the microarray. Expression levels in different samples and conditions may now be compared using a variety of statistical methods.

Although microarrays may be used in certain embodiments, various other methods of detection of gene expression are available. This section describes a few exemplary methods for detecting and quantifying mRNA or polypeptide encoded thereby.

In one embodiment, mRNA obtained from a sample is reverse transcribed into a first cDNA strand and subjected to PCR, e.g., RT-PCR. House keeping genes, or other genes whose expression does not vary may be used as internal controls and controls across experiments. Following the PCR reaction, the amplified products may be separated by electrophoresis and detected. By using quantitative PCR, the level of amplified product will correlate with the level of RNA that was present in the sample. The amplified samples may also be separated on an agarose or polyacrylamide gel, transferred onto a filter, and the filter hybridized with a probe specific for the gene of interest. Numerous samples may be analyzed simultaneously by conducting parallel PCR amplification, e.g., by multiplex PCR.

“Dot blot” hybridization has gained wide-spread use, and many versions were developed (see, e.g., M. L. M. Anderson and B. D. Young, in Nucleic Acid Hybridization-A Practical Approach, B. D. Hames and S. J. Higgins, Eds., IRL Press, Washington D.C., Chapter 4, pp. 73-111, 1985).

In another embodiment, mRNA levels is determined by dot blot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples may be analyzed simultaneously, since a blot may comprise multiple spots of RNA. Hybridization is detected using a method that depends on the type of label of the probe. In another dot blot method, one or more probes are attached to a membrane, and the membrane is incubated with labeled nucleic acids obtained from and optionally derived from RNA of a cell or tissue of a subject. Such a dot blot is essentially an array comprising fewer probes than a microarray.

Another format, the so-called “sandwich” hybridization, involves covalently attaching oligonucleotide probes to a solid support and using them to capture and detect multiple nucleic acid targets (see, e.g., M. Ranki et al. (1983) Gene, 21:77-85; A. M. Palva, et al, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. (1979) Nucleic Acid Res. 6, 11:3543; and B. J. Connor et al. (1983) PNAS 80:278-282). Multiplex versions of these formats are called “reverse dot blots.”

mRNA levels may also be determined by Northern blots. Specific amounts of RNA are separated by gel electrophoresis and transferred onto a filter which is then hybridized with a probe corresponding to the gene of interest. This method, although more burdensome when numerous samples and genes are to be analyzed provides the advantage of being very accurate.

Another method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that may be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388.

The level of expression of a biomarker may be determined by in situ hybridization. In one embodiment, a tissue sample is obtained from a subject, the tissue sample is sliced, and in situ hybridization is performed according to methods known in the art, to determine the level of expression of the genes of interest.

In other methods, the level of expression of a biomarker detected by measuring the level of protein encoded by biomarker gene. This may be done, e.g., by immunoprecipitation, ELISA, or immunohistochemistry using an agent, e.g., an antibody, that specifically detects the protein encoded by the gene. Other techniques include Western blot analysis. Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In the case of polypeptides which are secreted from cells, the level of expression of these polypeptides may be measured in biological fluids.

The above-described methods may be performed using cells grown in cell culture, or on cell or tissue specimens from a subject. Specimens may be obtained from an individual to be tested using either “invasive” or “non-invasive” sampling means. A sampling means is said to be “invasive” if it involves the collection of nucleic acids from within the skin or organs of an animal (including, especially, a murine, a human, an ovine, an equine, a bovine, a porcine, a canine, or a feline animal). Examples of invasive methods include blood collection, semen collection, needle biopsy, pleural aspiration, umbilical cord biopsy, etc. Examples of such methods are discussed by Kim, C. H. et al. (1992) J. Virol. 66:3879-3882; Biswas, B. et al. (1990) Annals NY Acad. Sci. 590:582-583; Biswas, B. et al. (1991) J. Clin. Microbiol. 29:2228-2233. It is also possible to obtain a cell sample from a subject, and then to enrich it in the desired cell type. For example, cells may be isolated from other cells using a variety of techniques, such as isolation with an antibody binding to an epitope on the cell surface of the desired cell type.

In certain embodiments, a single cell is used in the analysis. It is also possible to obtain cells from a subject and culture the cells in vitro, such as to obtain a larger population of cells from which RNA may be extracted. Methods for establishing cultures of non-transformed cells, i.e., primary cell cultures, are known in the art.

In other embodiments, the cell comprises a cell culture pellet, which may be present on a cell culture pellet microarray.

When analyzing from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA and proteins in the tissue and cells may quickly become degraded. Accordingly, in a preferred embodiment, the cells obtained from a subject are snap frozen as soon as possible.

In certain embodiments, a tissue sample or tissue section is present on a microarray. Paraffin-embedded formalin-fixed specimens may be prepared using punch “biopsy” cores from tissue specimens of interest. Each core may be arrayed into a separate recipient block, and sections cut and processed, for example as previously described in Konenen, J. et al., Tissue microarrays for high-throughput molecular profiling of tumor specimens, (1987) Nat. Med. 4:844-7 and Chung, G. G. et al., Clin Cancer Res. (2001) December; 7(12):4013-20.

C. Methods for Comparing Test Values with a Reference Set

Comparison to a reference set is particularly useful in applications of the above-described methods, for example when they are used in methods for diagnosing and prognosing a breast cancer in a subject, or for selecting therapeutics for a subject having breast cancer. The data obtained thereby, for example a ratio, or AQUA™ scores, may further be compared to a reference set of values associated with various states of breast cancer, various treatment outcomes, survival rates, etc.

Comparison of the determined value with reference values is preferably conducted using computer systems. In one embodiment, a ratio, AQUA™ scores, or other measures of protein amount are obtained in two cells and the values from the two cells are introduced into a computer system for comparison. In a preferred embodiment, one value is entered into a computer system for comparison with values that are already present in the computer system, or in computer-readable form that is then entered into the computer system.

In one embodiment, the invention provides computer readable forms of HER2/ER ratios, AQUA™ scores, or other measures of protein amount.

The data may be in the form of a table, such as an Excel table. The data may be alone, or it may be part of a larger database, e.g., comprising other expression profiles. For example, the data may be part of a public database. The computer readable form may be in a computer.

In one embodiment, the invention provides methods for determining the similarity between the HER2/ER ratio, AQUA™ score, or other measure of gene expression in a first cell, e.g., a cell of a subject, and that in a second cell, comprising obtaining the HER2/ER ratio, AQUA™ score, or other measure of gene expression in a first cell and entering these values into a computer comprising a database including records comprising values corresponding to the HER2/ER ratio, AQUA™ score, or other measure of gene expression in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing the HER2/ER ratio, AQUA™ score, or other measure of gene expression are entered into a computer system, comprising one or more databases with reference HER2/ER ratios, AQUA™ scores, or other measures of protein amount obtained from more than one cell. For example, a computer may comprise expression data of diseased and normal cells. Instructions are provided to the computer, and the computer is capable of comparing the data entered with the data in the computer to determine whether the data entered is more similar to that of a normal cell or of a diseased cell.

In another embodiment, the computer comprises the HER2/ER ratio, AQUA™ score, or other measure of gene expression in cells of subjects at different stages of breast cancer and the computer is capable of comparing the HER2/ER ratio, AQUA™ score, or other measure of gene expression entered into the computer with the data stored, and produce results indicating to which of the HER2/ER ratio, AQUA™ score, or other measure of gene expression in the computer, the one entered is most similar to the determined HER2/ER ratio, AQUA™ score, or other measure of protein amount, such as to determine the stage of cancer in the subject.

In yet another embodiment, the reference expression profiles in the computer are the HER2/ER ratio, AQUA™ score, or other measure of gene expression from cells of one or more subjects having breast cancer, which cells are treated in vivo or in vitro with a drug used for therapy of breast cancer. Upon entering the HER2/ER ratio, AQUA™ score, or other measure of gene expression of a cell of a subject treated in vitro or in vivo with the drug, the computer is instructed to compare the data entered to the data in the computer, and to provide results indicating whether the data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides systems comprising a means for receiving the HER2/ER ratio, AQUA™ score, or other measure of gene expression for one or a plurality of samples; a means for comparing the HER2/ER ratio, AQUA™ score, or other measure of gene expression from each of said one or plurality of samples to a common reference frame; and a means for presenting the results of the comparison. A system may further comprise a means for clustering the data.

In another embodiment, the invention provides computer programs for analyzing the HER2/ER ratio, AQUA™ score, or other measure of gene expression comprising (a) a computer code that receives as input the HER2/ER ratio, AQUA™ score, or other measure of gene expression for a plurality of samples and (b) a computer code that compares the HER2/ER ratio, AQUA™ score, or other measure of gene expression from each of said plurality of samples to a common reference frame.

The invention also provides machine-readable or computer-readable media including program instructions for performing the following steps: (a) comparing a plurality of values corresponding to the HER2/ER ratio, AQUA™ score, or other measure of gene expression in a query cell with a database including records comprising reference HER2/ER ratios, AQUA™ scores, or other measures of protein amount of one or more reference cells and an annotation of the type of cell; and (b) indicating to which cell the query cell is most similar based on similarities of the HER2/ER ratio, AQUA™ score, or other measure of protein amount. The reference cells may be cells from subjects at different stages of breast cancer or with different prognoses, for example. The reference cells may also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides methods for selecting a therapy for a patient having breast cancer; the methods comprising: (a) providing the HER2/ER ratio, AQUA™ score, or other measure of gene expression in a diseased cell of the patient; (b) providing a plurality of reference HER2/ER ratios, AQUA™ scores, or other measures of protein amount, each associated with a therapy, wherein the subject expression profile and each reference profile has a plurality of values, each value representing a HER2/ER ratio, AQUA™ score, or other measure of protein amount; and (c) selecting the reference profile most similar to the subject expression profile, to thereby select a therapy for said patient. In a preferred embodiment step (c) is performed by a computer. The most similar reference profile may be selected by weighing a comparison value of the plurality using a weight value associated with the corresponding expression data.

A computer readable medium may further comprise a pointer to a descriptor of a stage of breast cancer or to a treatment for breast cancer.

In operation, the means for receiving the HER2/ER ratio, AQUA™ score, or other measure of protein amount, the means for comparing the HER2/ER ratio, AQUA™ score, or other measure of protein amount, the means for presenting, the means for normalizing, and the means for clustering within the context of the systems of the present invention may involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that may cause a computer to function in the particular fashion described herein.

Those skilled in the art will understand that the systems and methods of the present invention may be applied to a variety of systems, including IBM®-compatible personal computers running MS-DOS® or Microsoft Windows®.

Exemplary diagnostic tools and assays are set forth below, which comprise the above-described methodology.

In one embodiment, the invention provides methods for determining whether a subject has or is likely to develop breast cancer, comprising determining the HER2/ER ratio, AQUA™ score, or other measure of gene expression in a cell of the subject and comparing the HER2/ER ratio, AQUA™ score, or other measure of gene expression with the HER2/ER ratio, AQUA™ score, or other measure of gene expression in a diseased cell of a subject known to have a disorder associated with cellular dedifferentiation, such that a similar the HER2/ER ratio, AQUA™ score, or other measure of gene expression is indicative that the subject has or is likely to develop breast cancer. In a preferred embodiment, the cell is essentially of the same type as that which is diseased in the subject.

In another embodiment the HER2/ER ratio, AQUA™ score, or other measure of gene expression may be used to confirm that a subject has a specific type or stage of breast cancer, and not a related disease or disease with similar symptoms. This may be important, in particular, in designing an optimal therapeutic regimen for the subject. Such distinction is known in the art as “differential diagnosis”.

In yet another embodiment, the invention provides methods for determining the stage of a breast cancer. It is thought that the HER2/ER ratio, AQUA™ score, or other measure of gene expression correlates with the stage of the disease. This could be confirmed, e.g., by analyzing the HER2/ER ratio, AQUA™ score, or other measure of gene expression in subjects having breast cancer at different stages, as determined by traditional methods. For example, the HER2/ER ratio, AQUA™ score, or other measure of gene expression of a diseased cell in subjects at different stages of the disease may be determined as described herein. Then, to determine the stage of breast cancer in a subject, the HER2/ER ratio, AQUA™ score, or other measure of gene expression in the subject is determined. A similar level of expression of the HER2/ER ratio, AQUA™ score, or other measure of gene expression between that in a subject and that in a reference profile of a particular stage of the disease, indicates that the breast cancer of the subject is at the particular stage.

Similarly, the methods may be used to determine the stage of the disease in a subject undergoing therapy, and thereby determine whether the therapy is effective. Accordingly, in one embodiment, the HER2/ER ratio, AQUA™ score, or other measure of gene expression is determined in a subject before the treatment and several times during the treatment. For example, a sample may be obtained from the subject before the beginning of the therapy and every 12, 24 or 72 hours during the therapy. Samples may also be analyzed once a week or once a month. Changes in the HER2/ER ratio, AQUA™ score, or other measure of gene expression over time and relative to diseased cells and normal cells will indicate whether the therapy is effective.

In yet another embodiment, the invention provides methods for determining the likelihood of success of a particular therapy in a subject having breast cancer. In one embodiment, a subject is started on a particular therapy, and the effectiveness of the therapy is determined, e.g., by determining the HER2/ER ratio, AQUA™ score, or other measure of gene expression in a cell of the subject. A normalization of the HER2/ER ratio, AQUA™ score, or other measure of gene expression, indicates that the treatment should be effective in the subject.

Prediction of the outcome of a treatment in a subject may also be undertaken in vitro. In one embodiment, cells are obtained from a subject to be evaluated for responsiveness to the treatment, and incubated in vitro with the therapeutic drug. The HER2/ER ratio, AQUA™ score, or other measure of gene expression is then measured in the cells and these values are compared to the HER2/ER ratio, AQUA™ score, or other measure of gene expression in a cell which is the normal counterpart cell of a diseased cell. The HER2/ER ratio, AQUA™ score, or other measure of gene expression may also be compared to that in a normal cell. The comparative analysis is preferably conducted using a computer comprising a database of the HER2/ER ratio, AQUA™ score, or other measure of gene expression as described above. A HER2/ER ratio, AQUA™ score, or other measure of gene expression ratio in the cells of the subject after incubation with the drug that is similar to the HER2/ER ratio, AQUA™ score, or other measure of gene expression in a normal cell and different from that in a diseased cell may indicate that the subject is likely to respond positively to a treatment with the drug. On the contrary, a HER2/ER ratio, AQUA™ score, or other measure of gene expression in the cells of the subject after incubation with the drug that is similar to the HER2/ER ratio, AQUA™ score, or other measure of gene expression in a diseased cell and different from that in a normal cell may indicative that the subject is unlikely to respond positively to a treatment with the drug.

Since it is possible that a drug does not act directly on the diseased cells, but is, e.g., metabolized, or acts on another cell which then secretes a factor that will effect the diseased cells, the above assay may also be conducted in a tissue sample of a subject, which contains cells other than the diseased cells. For example, a tissue sample comprising diseased cells can be obtained from a subject; the tissue sample can then be incubated with the potential drug; optionally one or more diseased cells can be isolated from the tissue sample, e.g., by microdissection or Laser Capture Microdissection (LCM, see infra); and the HER2/ER ratio can be examined.

The invention may also provide methods for selecting a therapy for a breast cancer patient. For example, certain subjects having breast cancer may respond better to one type of therapy than another type. In a preferred embodiment, the method comprises comparing the HER2/ER ratio, AQUA™ score, or other measure of gene expression in the patient with that in cells of subjects treated in vitro or in vivo with one of several therapeutic drugs, which subjects are responders or non responders to one of the therapeutic drugs, and identifying the cell which has the most similar HER2/ER ratio, AQUA™ score, or other measure of gene expression to that of the patient, to thereby identify a therapy for the patient. The method may further comprise administering the identified therapy to the subject.

E. Kits

The present invention provides kits for practice of the afore-described methods. In certain embodiments, kits may comprise antibodies against HER2, ER, epidermal growth factor receptor (EGFR), other HER family members such as HER1, HER 2, HER3 and HER4, progesterone receptor (PR), GATA3 and NAT1. In other embodiments, a kit may comprise appropriate reagents for determining the level of protein activity in the cells of a subject.

In still other embodiments, a kit may comprise a microarray comprising probes of HER2, ER epidermal growth factor receptor (EGFR), other HER family members such as HER1, HER 2, HER3 and HER4, progesterone receptor (PR), GATA3 and NAT1 genes or proteins. A kit may comprise one or more probes or primers for detecting the expression level of HER2, ER, epidermal growth factor receptor (EGFR), other HER family members such as HER1, HER 2, HER3 and HER4, progesterone receptor (PR), GATA3 and NAT1 and/or a solid support on which probes are attached and which may be used for detecting expression. A kit may further comprise controls, buffers, and instructions for use.

Kits may also comprise a library of HER2/ER ratios associated with survival, response to therapy, stage of disease, etc., e.g., reference sets.

In one embodiment, the kit comprises a computer readable medium on which is stored one or more HER2/ER ratios, AQUA™ scores, or other measures of gene expression associated with survival, response to therapy, stage of disease, etc., or at least values representing HER2/ER ratios, AQUA™ scores, or other measures of gene expression associated with survival, response to therapy, stage of disease, etc. The kit may comprise ratio analysis software capable of being loaded into the memory of a computer system. Kit components may be packaged for either manual or partially or wholly automated practice of the foregoing methods. In other embodiments involving kits, this invention contemplates a kit including compositions of the present invention, and optionally instructions for their use. Such kits may have a variety of uses, including, for example, imaging, diagnosis, therapy, and other applications.

In addition to the embodiments, aspects and objects of the present invention disclosed herein, including the claims appended hereto, the following paragraphs set forth additional, non-limiting embodiments and other aspects of the present invention (with all references to paragraphs contained in this section referring to other paragraphs set forth in this section):
1. A method of determining whether a subject having breast cancer is likely to respond to a breast cancer therapy, comprising determining the ratio of HER2 expression to ER expression in a cell of said subject, wherein the determined ratio indicates whether the subject is likely to respond to the breast cancer therapy.
2. The method of paragraph 1, wherein the breast cancer therapy is selected from the group consisting of: HER2-based therapy and hormone therapy.
3. A method of selecting a breast cancer therapy for a subject, comprising determining the ratio of HER2 expression to ER expression in a cell of said subject, wherein the determined ratio indicates the appropriate therapy for the subject.
4. The method of paragraph 1, which further comprises determining the expression level of epidermal growth factor receptor (EGFR).
5. The method of paragraph 1, which further comprises determining the expression level of a member selected from the group consisting of HER1, HER2, HER3, HER4, PR, GATA3 and NAT1.
6. The method of paragraph 1, wherein the HER2-based therapy is Herceptin™.
7. A method of diagnosing or prognosing breast cancer in a subject, comprising determining the ratio of HER2 expression to ER expression in a cell of said subject, wherein the determined ratio indicates the presence or stage of breast cancer in a subject and allows a prediction of the subject's chance of survival.
8. The method of paragraph 1, wherein determining the ratio HER2 expression to ER expression comprises determining the level of expression of the genes encoding HER2 and ER in the cell.
9. The method of paragraph 1, wherein determining the ratio of HER2 expression to ER expression comprises determining the amounts of HER2 and ER protein in the cell.
10. The method of paragraph 9, wherein the amounts of HER2 and ER protein are determined using antibodies against the HER2 and ER proteins.
11. The method of paragraph 8, wherein the level of expression of HER2 and ER is determined by determlining the AQUA™ score of each of HER2 and ER.
12. The method of paragraph 11, wherein the AQUA™ score of each of HER2 and ER is determined using the AQUA™ automated pathology system.
13. The method of paragraph 1, wherein the ratio of HER2 expression to ER expression is determined using a microarray.
14. The method of paragraph 13, wherein the microarray comprises nucleic acids that hybridize to the HER2 and ER gene or gene product.
15. The method of paragraph 13, wherein the microarray comprises polypeptides that bind to HER2 and ER.
16. The method of paragraph 1, wherein the cell comprises a tissue sample.
17. The method of paragraph 16, wherein the tissue sample is present on a microarray.
18. The method of paragraph 1, wherein determining the ratio of HER2 expression to ER expression comprises determining the location of HER2 and ER expression in the cell.
19. A kit comprising reagents for detecting HER2 and ER.
20. The kit of paragraph 19, further comprising instructions for use.
21. A method of determining whether a subject having breast cancer is likely to respond to a breast cancer therapy, comprising quantifying the expression level in an appropriate cell of all of ER, GATA3, NAT1 and/or quantifying the expression level of at least two of EGFR, HER1, HER2, HER3, HER4, ER and PR, in a cell of said subject, wherein the determined ratio and/or quantified expression level indicates whether the subject is likely to respond to the breast cancer therapy.
22. The method of paragraph 21, wherein the breast cancer therapy is selected from the group consisting of: HER2-based therapy and hormone therapy.
23. A method of selecting a breast cancer therapy for a subject, comprising quantifying the expression level of all of ER, GATA3, NAT1 and/or quantifying the expression level of at least two of EGFR, HER1, HER2, HER3, HER4, ER and PR, in a cell of said subject, wherein the determined ratio or expression level indicates the appropriate therapy for the subject.
24. The method of paragraph 21, wherein the breast cancer therapy is selected from either HER2-based therapy or hormone therapy.
25. The method of paragraph 22, wherein the determined ratio indicates that the subject is likely to respond to HER2-based therapy but not hormone therapy.
26. The method of paragraph 22, wherein the determined ratio indicates that the subject is likely to respond to hormone therapy but not HER2-based therapy.
27. A method of diagnosing or prognosing breast cancer in a subject, comprising quantifying the expression level of all of ER, GATA3, NAT1 and/or quantifying the expression level of at least two of EGFR, HER1, HER2, HER3, HER4, ER and PR, in a cell of said subject, wherein the determined ratio or expression level indicates the presence or stage of breast cancer in a subject and allows a prediction of the subject's chance of survival.
28. The method of any one of paragraphs 21, 23 or 27, wherein quantifying the expression level of ER, GATA3, NAT1, EGFR, HER1, HER2, HER3, HER4, ER and PR comprises quantifying the level of expression of the genes ER, GATA3, NAT1, EGFR, HER1, HER2, HER3, HER4, 2C4, ER and PR.
29. The method of any one of paragraphs 21, 23 or 27, wherein quantifying the expression level of ER, GATA3, NAT1, EGFR, HER1, HER2, HER3, HER4, 2C4, ER and PR comprises quantifying the amounts of ER, GATA3, NAT1, EGFR, HER1, HER2, HER3, HER4, 2C4, ER and PR protein in the cell.
30. The method of any one of paragraphs 29 or 31, wherein the amounts of protein are quantified using antibodies against the proteins.
31. The method of paragraph 30, wherein the level of expression is quantified by determining the AQUA™ score of ER, GATA3, NAT1, EGFR, HER1, HER2, HER3, HER4, 2C4, ER and/or PR.
32. The method of paragraph 31, wherein the AQUA™ score of ER, GATA3, NAT1, EGFR, HER1, HER2, HER3, HER4, 2C4, ER and/or PR is determined using the AQUA™ automated pathology system.
33. The method of paragraph 30, wherein the level of expression is quantified using a microarray.
34. The method of paragraph 33, wherein the microarray comprises polypeptides that are able to detect ER, GATA3, NAT1, EGFR, HER1, HER2, HER3, HER4, ER and/or PR.
35. The method of paragraph 28, wherein the level of expression is quantified using a microarray.
36. The method of paragraph 35, wherein said tissue sample is present on a microarray.
37. The method of paragraph 28, wherein determining the level of expression comprises determining the location of ER, GATA3, NAT1, EGFR, HER1, HER2, HER3, HER4, ER and/or PR expression in the cell.
38. A kit comprising reagents for detecting ER, GATA3, NAT1, EGFR, HER1, HER2, HER3, HER4, 2C4, ER and/or PR.
39. The kit of paragraph 38, further comprising instructions for use.
40. A computer implemented method for selecting at least one biomarker for use in prognosing breast cancer, comprising:
   (a) defining an individual of a genetic algorithm construct having n chromosomes, wherein each chromosome represents at least one biomarker;
   (b) defining a fitness function based on desired attributes of the at least one biomarker;
   (c) selecting one or more of said individuals for inclusion in an initial population; and
   (d) executing a genetic algorithm on said population until defined convergence criteria are met, wherein execution of said genetic algorithm comprises the steps of:
   (i) choosing the fittest individual from said population;
   (ii) choosing random individuals from said population; and
   (iii) creating offspring from said fittest and said randomly chosen individuals.

### EXEMPLIFICATION

The present invention is further illustrated by the following examples which should not be construed as limiting in any way.

### Example 1

### HER2/ER Ratio as a Predictive Diagnostic Test

AQUA™ was used to measure the ratio of HER2 to ER biomarker expression using a 650 case retrospective breast cancer tissue microarray (TMA) looking at overall survival in a cohort from the 1960s and 1970s. The ratio normalizes for both individual variations in expression level as well as variations in tissue fixation and antigen retrieval. Table I shows that the ratio of HER2/ER was more predictive of survival than either biomarker alone:

TABLE I
Analysis of outcome on a 650 case breast cancer TMA with 10
year follow-up

| | Univariate Chi | Univariate p | Multivariate | Multivariate |
|---|---|---|---|---|
| Marker | Sq. | value | Chi Sq. | p value |
| HER2 | 9.6 | 0.0019 | 2.98 | 0.0843 |
| ER | 13 | 0.002 | 7.14 | 0.0076 |
| HER2/ER | 41 | <0.0001 | 13.7 | 0.0002 |

### Example 2

### HER2/ER Ratio in Predicting Response to Breast Cancer Therapy

The above-described ratio test may also be used to predict response to hormone therapies (like Tamoxifen or Aromatase inhibitors) or to HER2-based therapies (like Herceptin and Iressa) in breast cancer. The HER2/ER ratio may be measured in arrays from prospective clinical trials of hormonal and HER2-based therapies in order to establish its correlation with the outcome of such therapies in subjects having various types and stages of breast cancer.

### Example 3

### Multiplexed HER Test as a Predictive Diagnostic Test for Herceptin™

A series of antibodies were tested to all of the classical members of the ErbB2 (HER) family. Specifically, using AQUA™, the expression of HER1, HER2, HER3, HER4 were tested and the information combined with data from 2C4 (a HER2 mouse monoclonal antibody), ER and PR.

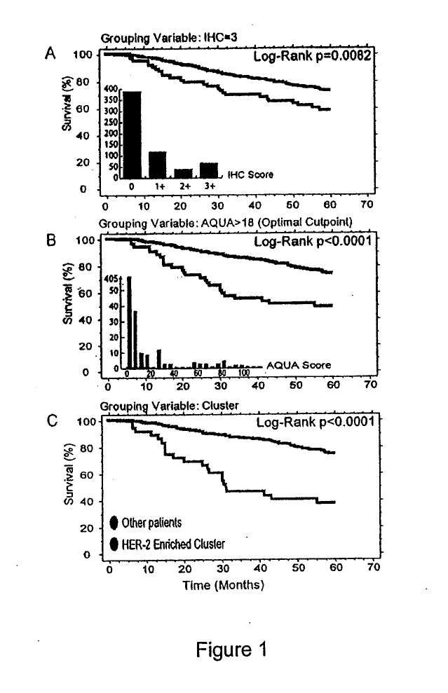

FIG. 1 shows the improvement of prognostic value in a historical cohort of nearly 500 breast cancer cases. The conventional approach defines a subset of patients with high (3+) expression that have a 5-year survival of 55%. By quantitative analysis of HER2, a more precise group of patients that show a 5-year survival of 47% were defined. Finally, it has been shown that multiplexing a set of HER family proteins defines a subset (about ¾) of the patients that are HER2 positive as defined by a pathologist. That subset has a 36% 5-year survival. In each of these cases, the negative cases have a 5-year survival between 70% and 78%.

### Example 4

### Multiplexed “Alliance Assay” as a Predictive Diagnostic Test for Herceptin™

AQUA™-based objective quantitative analysis of tissue microarrays has been used to discover a minimal multiplexed set of tissue biomarkers, including GATA3, NAT1 and ER, that can be applied to conventional formalin fixed, paraffin embedded tissue sections. Genetic algorithms (as further described in Example 5) were then applied on a cohort of 502 breast cancer patients to discover a prospectively applicable solution that can define a subset of patients with 5 year survival of 96% which was validated on an independent cohort from Sweden. This test has both the familiarity and specificity for widespread use in management of breast cancer.

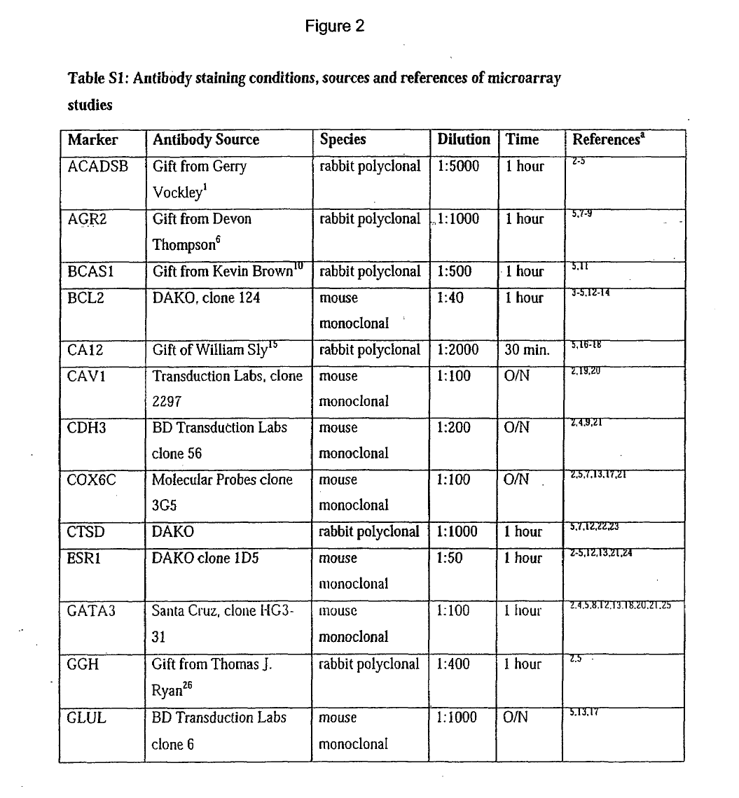

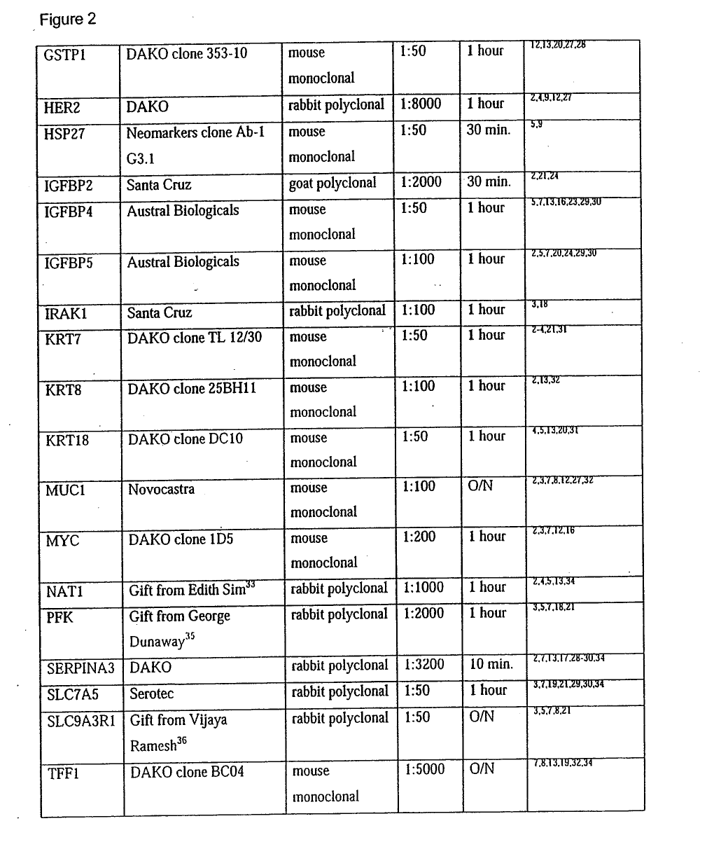

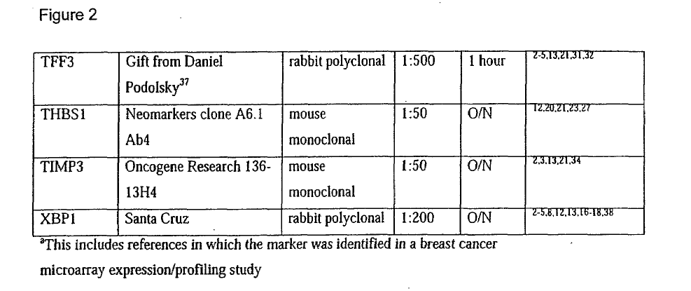

Antibodies to thirty-five markers that were either well-characterized commercial antibodies or well-studied antibodies from individual labs were obtained (FIG. 2). A 250-case breast cancer cohort was then assessed by AQUA™ to quantify the expression of each marker in the epithelial tumor regions of the tissue microarray histospots. The resulting scores are measurements of the biomarker pixel intensity within a compartment divided by the total area of epithelium to account for differences in epithelial area in each spot. The generation of continuous data for each marker allowed objective assessment of protein expression and use of the data in rigorous algorithmic analyses.

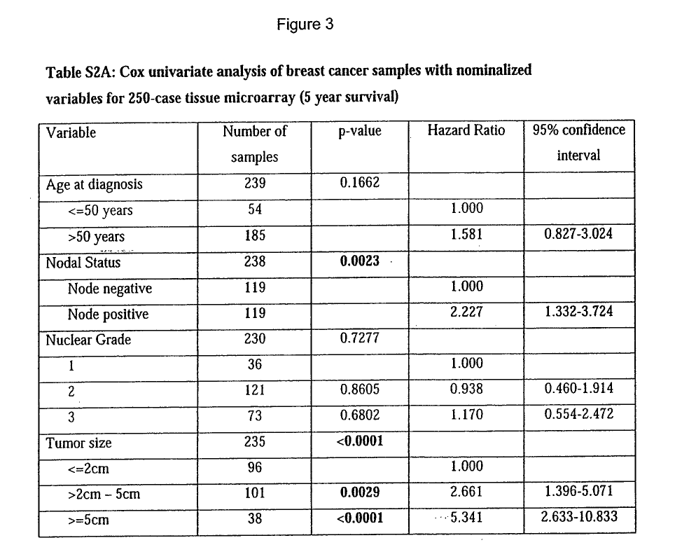

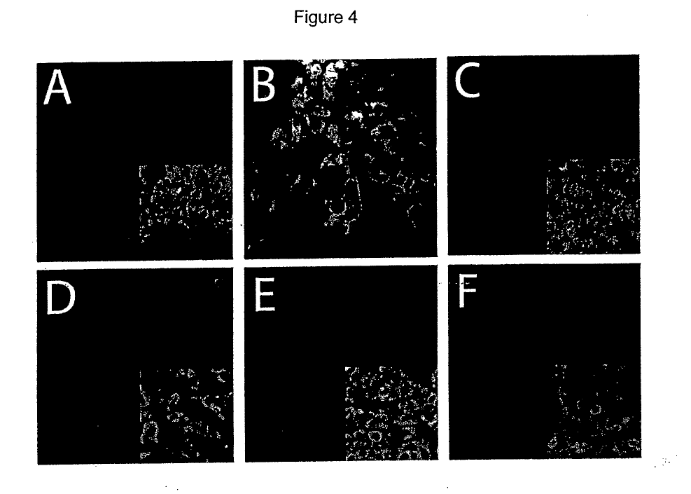

To further analyze each marker, the survival and disease-specific death information for the cohort was utilized to investigate the relationship of each marker with prognosis by univariate Cox proportional hazards (FIG. 3). Six of the thirty-five markers were significant when assessed by their continuous protein expression levels for relationship with 5 year disease specific surgical, including BCL2 (p=0.0400), COX6C (p=0.0049), ER (p=0.0115), GATA3 (p=0.0011), HER2 (p=0.0048) and NAT1 (p=0.0206). Examples of immunofluorescence staining are shown in FIG. 4 for high protein expression of ER, NAT1, and GATA3 (FIG. 4A, 4B, 4C respectively) and for very low expression of ER, NAT1 and GATA 3 (FIG. 4C, 4D, 4E respectively).

While the individual prognostic value of markers is important, the classification strength from array studies derives from the power of multiplexing. Since the markers were selected based on previous expression profiling studies (FIG. 2) and because continuous data was generated for each marker, the methods of hierarchical clustering and visualization of a heat map were applied to provide such a context for exploration of the biological co-expression. This type of expression analysis was done to identify markers that had the highest correlation with ER protein levels, but that may still provide additional classification or predictive power under further examination.

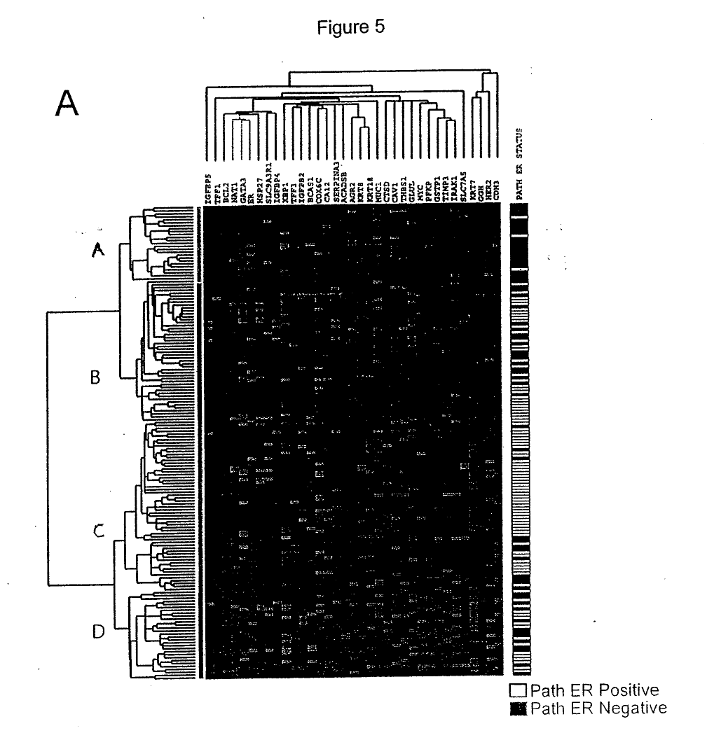

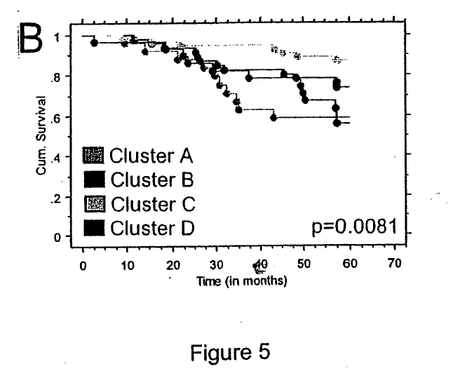

Unsupervised average linkage clustering was applied to this set of markers as shown in the heat map in FIG. 5A. Estrogen receptor is in a small cluster with two other markers, GATA3 and NAT1. The larger cluster containing the GATA3, NAT1 and ER grouping also includes BCL2, HSP27, SLC9A3R1 and IGFBP4. When looking at the results of linear regression analysis for these markers individually with estrogen receptor protein expression levels, they had statistically significant direct relationships (i.e. higher ER levels corresponding to higher marker levels) as follows: BCL2 (R=0.423, p<0.0001), GATA3 (R=0.497, p<0.0001), HSP27 (R=0.327, p<0.0001), IGFBP4 (R=0.203, p=0.0055), NAT1 (R=0.487, p<0.0001) and SLC9A3R1 (R=0.187, p=0.0182). There was a noticeable grouping of markers (CDH3, KRT7, GGH and HER2) away from the rest of the markers in the heat map that generally have indirect relationships with estrogen receptor levels (i.e. higher ER levels correspond to lower levels). Also to be noted is the close relationship in the clustering analysis of keratin 8 and 18, which are known to be co-expressed.

The cluster with the highest estrogen receptor levels, Cluster C, had the best prognosis and Cluster A, with the lowest ER levels, had a poor prognosis (87.5% and 60.0% 5-year survival, respectively) (FIG. 5A, 5B). When comparing the cluster groupings with traditional immunohistochemistry determined ER status, there were differences in ER status positivity between the groupings. Cluster C consists of 81% ER-positive tumors, Cluster B with 56% ER positive tumors, Cluster D with 53% ER positive tumors, and Cluster A with 12% ER positive tumors. This analysis shows that multiplex expression analysis can reveal prognostic classes that are substantially different from those obtained with current immunohistochemical methods.

Since the ultimate goal of this discovery was to find a minimal number of markers with maximal prognostic or predictive value, a small grouping was focused on two markers, GATA3 and NAT1, with high correlation with ER protein expression (highlighted in FIG. 5A). As shown in the univariate Cox proportional hazards analyses in FIG. 3, higher levels of ER, GATA3, and NAT1 are all individually related to better patient prognosis.

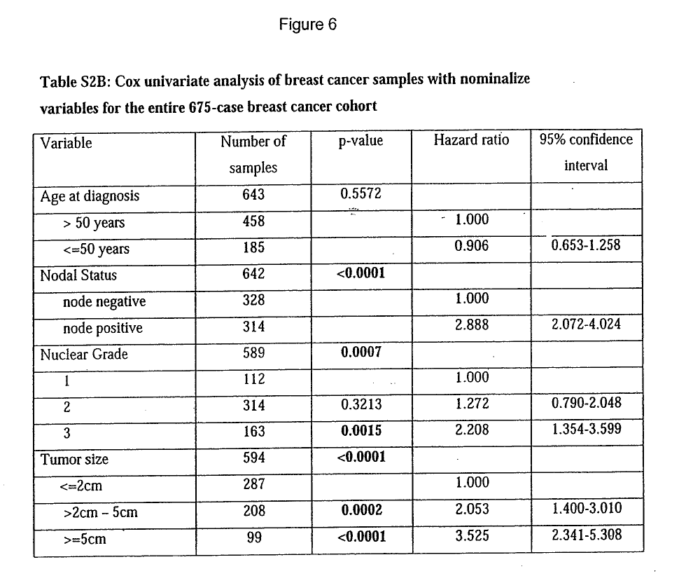

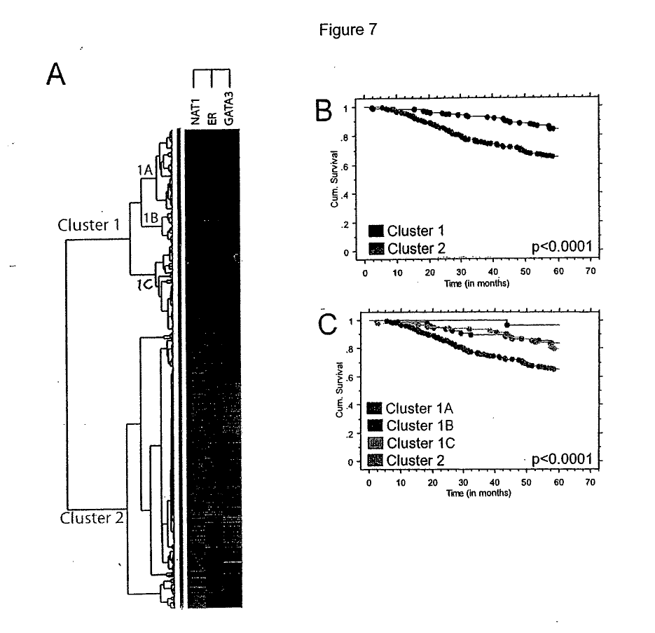

To more rigorously evaluate the three markers, AQUA™ analysis for ER, GATA3 and NAT1 was performed on an expanded set of breast cancer tumors on tissue microarrays (cohort details in FIG. 6). Pairwise evaluation of these markers by Cox multivariate analysis methods shows each maintains independent predictive value. The results of unsupervised hierarchical clustering of ER, GATA3 and NAT1 for all of the breast cancer tumors defined two main clusters, Cluster 1 with different subsets of coexpression of NAT1, GATA3 and ER, and Cluster 2 with predominantly low levels of the three markers (FIG. 7). These two clusters had significant differences in prognosis as shown in FIG. 7B with 84.6% 5 year survival in Cluster 1 (log rank p<0.0001) versus 65.9% in Cluster 2. While Cluster 2 demonstrates remarkably homogeneity in expression of the three markers, Cluster 1 illustrates that while having overall higher levels ER, NAT1 and GATA3, there are groupings of marker co-expression that subclassify patients with good outcome (FIG. 7C). Specifically, one cluster (1 B) shows a 97% 5 year survival compared to another (1 C) with only 80%. Although the unsupervised clustering can define classes that represent dramatic differences in outcome, the method does not lend itself to prospective assignment of class, nor does it take advantage of outcome-based information in class discovery. Another method was sought for analysis of this three marker set for potential prospective use, genetic algorithm analysis, which is further described in Example 5.

There is evidence that suggests that GATA3 and NAT1 may be useful in combination with estrogen receptor status for fine-tuning prediction of hormonal response. Downregulation of GATA3 was seen by tamoxifen treatment in a microarray comparison of the tamoxifen sensitive mammary carcinoma xenograft MaCa 3366 and its tamoxifen resistant derivative MaCa 3366/TAM56. A small pilot study by Parikh et al 57 of two age matched groups of ER positive invasive breast cancer tumors with different responses to tamoxifen showed that the tamoxifen responsive group had higher mean levels of GATA3. Loss of GATA3 expression was significantly associated with an increased risk of hormone unresponsiveness when assessed on a sliding scale without choosing an optimal cutpoint. There are a few studies that suggest that NAT1 may be important in tamoxifen treatment of ER-positive breast cancer tumors as well. Greater inhibition of NAT1 enzyme activity has been seen after tamoxifen treatment in ER positive tumors than in ER negative tumors in a study of 30 breast cancer patients 58. Bieche et al 59 looked at the relationship between RNA expression levels of xenobiotic metabolizing enzymes by RT-PCR and benefit from adjuvant tamoxifen therapy. NAT1 RNA was elevated in ER positive tumors compared to normal breast tissue, and had a strong correlation with ER alpha mRNA levels in their pilot study. Their examination of a larger cohort of 97 breast cancer tumors showed that increased NAT1 mRNA levels corresponded with better prognosis, and that NAT1 was the most discriminatory of the enzymes between tamoxifen responders and non-responders.

Methods used in Example 4

Tissue Microarray Construction

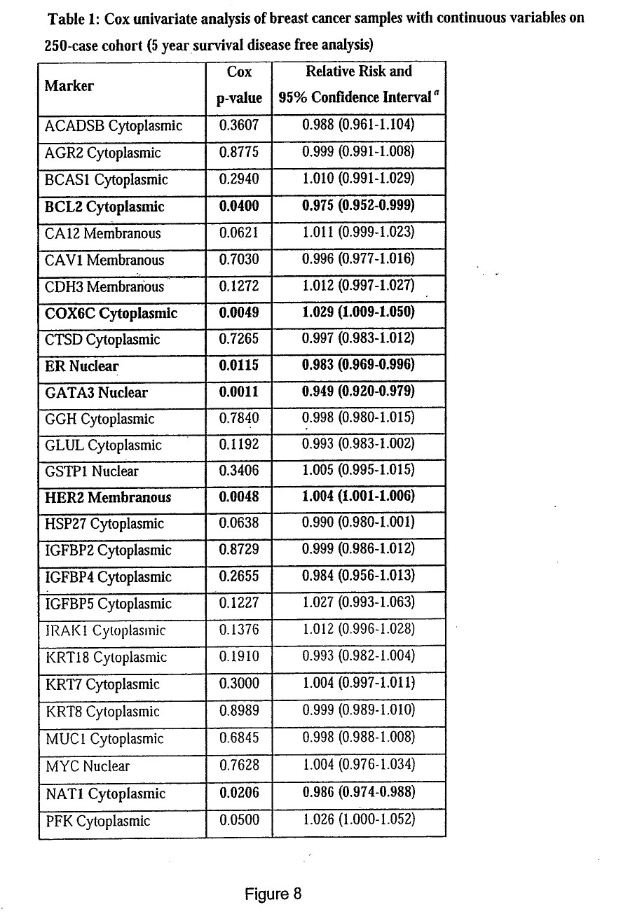

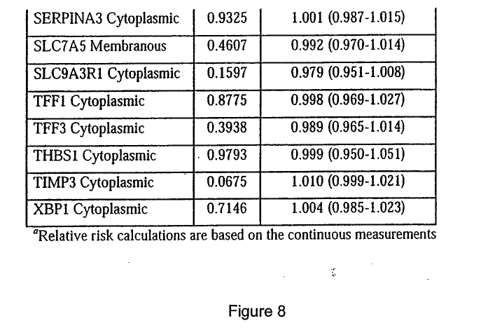

Formalin-fixed paraffin embedded breast cancer tumors from the archives of the Yale University Department of Pathology were utilized in the construction of the tissue microarrays. Briefly, representative areas of invasive ductal carcinoma were selected from whole tissue H&E sections by pathologists. Cores sized 0.6 mm in diameter were spaced 0.8 mm apart in a grid layout using a manual Tissue Microarrayer (Beecher Instruments, Silver Spring, Md.). The resulting tissue microarray blocks were cut to 51 μm sections with a microtome, placed on slides with an adhesive tape-transfer method (Instrumedics, Inc., Hackensack, N.J.), and UV crosslinked. The analysis of the thirty-five markers was all done on a 250-case cohort 10 of half node positive and half node negative specimens; the associations with clinical and pathological parameters with patient outcome are displayed in FIG. 8. There were 105 classified as ER negative (44.9%) and 129 cases classified as ER positive (55.1%) for those tumors in which the clinical ER status (as determined by immunohistochemical analysis) were available. Previous analysis from our lab has shown a high correlation between our AQUA™ scores and pathologist designated ER status. A larger cohort, utilized for further analysis of ER, GATA3 and NAT1, consists of a total of 675 tumors, including cores from the 250-case TMA tumors, with the additional samples consisting of 193 ER negative cases (48.3%) and 207 ER positive cases (51.8%) for those cases with available ER status information. The associations with clinical and pathological parameters with patient outcome for the entire cohort are displayed in FIG. 6. This entire cohort contains approximately half node-positive specimens and half node-negative specimens, and has also been used previously in other studies and is described in detail below. The cohort used for validation is from the control arm of a study from a randomized trial of Tamoxifen conducted in Sweden from 1986 to 1991. Details of this cohort are described below. In all cohorts, tissue microarray spots without sufficient breast tumor epithelium were excluded from the analysis.

Immunofluorescence Staining

The tissue microarrays were deparaffinized by two xylene rinses of 30 minutes each followed by two rinses with 100% ethanol for one minute each and a rinse in water. Antigen retrieval was performed by boiling the slides in a pressure cooker in a sodium citrate buffer at a pH of 6.0. After rinsing briefly in 1× Tris-buffered saline (TBS), a 30-minute incubation with 2.5% hydrogen peroxide/methanol was used to block endogenous peroxidases. In order to reduce nonspecific background staining, slides were incubated with 0.3% Bovine serum albumen (BSA)/1×TBS for one hour at room temperature, followed by a series of 2 minute rinses in 1×TBS, 1×TBS/0.01% Triton, 1×TBS (TBS washes). Slides were incubated overnight at 4° C. with either a monoclonal mouse anti-cytokeratin antibody (clone AE1/AE3, DAKO, Carpinteria, Calif., 1:100) when using a rabbit or goat target antibody, or a rabbit anti-cytokeratin antibody (1:100, DAKO) when using a mouse target antibody. The sources, dilutions and incubation times of the target antibodies are listed in FIG. 2. Antibodies were selected that had either been generated and validated by individual labs, or were well-characterized antibodies frequently used for immunohistochemistry in the literature. Slides were washed in 1×TBS rinses and incubated with secondary antibodies for 1 hour at room temperature as follows: Alexa 488 goat anti-rabbit (1:100, Molecular Probes, Eugene, Oreg.) for detecting cytokeratin, and species specific horseradish peroxidase (HRP) with a dextran-polymer backbone (Envision, DAKO) along with DAPI (6-diamidino-2-phenylindole, 1:100, DAKO) for visualization of nuclei. For goat primary antibody detection, slides were incubated for one hour with biotinylated anti-goat (1:200, Vector, Burlingame, Calif.) and Cy-2-donkey anti-mouse (1:30, Jackson Labs, Bar Harbor, Me.). This was followed by a one hour incubation with Streptavidin HRP (1:200, Perkin Elmer). All slides were washed with TBS rinses followed by a 10 minute incubation with Cy-5 tyramide (1:50 dilution in Amplification Diluent, Perkin Elmer). The slides were mounted in 0.6% n-propyl gallate (an anti-fade mounting medium) and coverslipped. Examples of immunofluorescent staining are shown in FIG. 4.

AQUA™ Analysis

AQUA™ software linked to a fluorescence microscopy system allowed for quantification of the protein of interest within the tumor region of each tissue microarray core. Image acquisition begins with a low resolution (64×64 pixels) image capture with a 10× objective. The rows and columns of the tissue microarray are defined to form a grid on which the histospots are placed. Monochromatic high resolution (1024×1024 pixels) in-focus and out-of-focus images are then captured for each relevant wavelength for each histospot. The pan-cytokeratin antibody separates the epithelial breast cancer tumor component by binary masking from the surrounding stroma, and other fluorescent tags designate subcellular compartments (i.e. DAPI for nuclei). Coalescence of cytokeratin staining was used to define the non-nuclear compartment for analysis of cytoplasmic and/or membranous staining. The images of the markers were captured at the Cy-5 wavelength because it is outside the range of tissue autofluorescence. Two algorithms, RESA and PLACE, were then used for AQUA analysis. RESA improves subcellular compartment assignment by taking into account overlapping of cells due to the thickness of the tissue microarray section on the slide by subtracting the in-focus information from the out-of-focus information. The second algorithm, PLACE, assigns target staining in Cy-5 to the pre-defined subcellular compartments and quantifies its intensity within each compartment in order to give a quantitative measurement of the expression level for each histospot. The resulting AQUA™ scores are the measurements of the biomarker pixel intensity within a compartment divided by the total area of tumor (to normalize for differences in tumor area in each spot), and were utilized as their raw values in the statistical analyses described below, unless otherwise noted. Tumor samples with more than one AQUA™ score for a given case were averaged.

Unsupervised Hierarchical Clustering

Z-score transformation was utilized to normalize between experiments in order to perform clustering analyses with this formula: [(AQUA score)−(mean of AQUA scores on TMA X)]/standard deviation, where X is a given TMA experiment assessed for a single marker. This has previously been utilized for analysis of AQUA scores. CLUSTER26 was used to perform unsupervised average linkage hierarchical clustering on unweighted z-score transformed AQUA data and visualized with TREEVIEW, as done previously. For the 35 marker clustering (FIG. 5A), cases shown include those with data for at least 80% of the markers to allow inclusion of a larger number of samples due to missing data; the three-marker clustering (FIG. 5B) only includes cases with data for all three markers.

Statistical Analyses

Statview 5.0.1 (SAS Institute, Inc., Cary, N.C.) was used for the Cox proportional hazards analysis. Clusters from the unsupervised hierarchical clustering analyses, and groups from the multiplex marker assay were assessed for relationship to patient prognosis by Kaplan-Meier survival curves with their significance analyzed by the Mantel-Cox log-rank test with Statview 5.0.1 or SPSS (SPSS Inc., Chicago, Ill.).

Detailed Information on Cohorts

The Yale breast cancer cohort consists of 688 samples of invasive ductal carcinoma selected from the Yale University Department of Pathology archives as available from 1961 to 1983. The mean follow-up time of this cohort is 12.8 years and the mean age of diagnosis is 58.1 years. The median follow-up time is 8.9 years and the median age of diagnosis is 58.0 years. Of the 656 patients with follow-up time, 328 were censored at 20 years and 276 were uncensored at 20 years. Of the 328 censored patients, their median follow-up was 21.4 years, with the minimum at 4.2 months. This cohort contains approximately half node-positive specimens and half node-negative specimens, which have been utilized previously in other studies from our laboratory. Complete clinical treatment information was not available for this cohort; however some limited information was available for the node positive patients. Most node negative patients were treated with local radiation, and none received Herceptin. About 15% of the node positive patients were treated with chemotherapy (primarily Adriamycin, cytoxan and 5-fluorouracil), and subsequently about 27% received tamoxifen (post 1978). The node negative patients were largely treated only by surgical resection.

The Swedish breast cancer cohort consists of 564 pre-menopausal patients with invasive breast cancer, UICC stage 2, enrolled from 1986 to 1991 at two centers in Sweden 45 The patients were randomized to receive adjuvant Tamoxifen (n=276) or no adjuvant treatment (n−288) after a primary surgical treatment. Less than 2% of the patients received other adjuvant treatment. The median follow up time was 13.9 years. The arms were well matched, both having a median age of 45 years old. Between 27 and 30% of each arm showed no nodal metastasis at diagnosis.

GATA3 is a member of a zinc-finger domain transcription factor family, and null mutations are embryonic lethal during development. Several RNA studies have identified correlations between GATA3 and ER expression, as well as several protein expression studies, but GATA3 is not regulated by estradiol in MCF-7 cells. GATA3 and ER are not coexpressed in a cDNA microarray analysis of ovarian tumors, suggesting that this relationship may be characteristic of breast cancer tumors. Prognostic assessment of GATA3 protein expression has shown that high expression of GATA3 is associated with good prognosis, which is in agreement with our results. An analysis of GATA3 mutations in 111 ER positive breast cancer tumors by Usary et al revealed five tumors with somatic mutations near a conserved zinc-finger domain. Further analysis by microarray analysis of ectopic wild-type GATA3 resulted in induction of several genes identified in the luminal epithelial ER positive RNA cluster and in differentiation of luminal cells, but did not include ER.

High levels of heterocyclic amines have been linked to increased breast cancer risk, so enzymes that can metabolize these compounds are important. NAT1 (n-acetyltransferase 1) is a Phase II drug metabolizing enzyme that catalyzes the transfer of an acetyl group from acetyl-CoA to the terminal nitrogen of heterocyclic and other carcinogenic amines, however there have been no significant associations of different NAT1 genotypes with increased breast cancer risk. NAT1 RNA expression has been strongly correlated with ER status and ER RNA expression in several studies. Protein level analyses have been limited. Proteome analysis of normal and breast cancer tissues by Adam et al identified NAT1 as consistently elevated in invasive breast cancer, with its enzyme activity being independent of stimulation by 17-beta estradiol and with a strong association of NAT1 cytoplasmic expression with ER positivity in 36 ER positive tumors. Increased NAT1 expression has been seen in well-differentiated bladder cancer suggesting that it functions primarily in that tumor type to detoxify and protect the bladder from harmful aromatic amine metabolites, rather than bioactivating the metabolites. The results of those studies are in agreement with the relationship we see with high NAT1 expression and ER, and good prognosis in our breast cancer tumors. In our cohort, NAT1 expression blanketed over both the cytoplasm and nuclei of the cells (FIG. 4B), but was predominately cytoplasmic. Due to this type of staining pattern, nuclear NAT1 expression was highly correlated with cytoplasmic expression, even though the vast majority of cases did not have strong distinct nuclear expression. We focused on cytoplasmic NAT1 expression due to its predominance in staining and because it was the subcellular localization used in previous studies of those utilizing the same lab as the source of the NAT1 antibody. Future investigations may be able to shed some light on the role of weak nuclear NAT1 expression in breast cancer in relation to estrogen receptor and its potential involvement in therapeutic response.

### Example 5

### Genetic Algorithm for Generation of a Multiplex Marker Assay

Genetic algorithm methods have been used extensively in other fields to discover a set of mathematical functions that best define properties of a population. They have been less commonly used in array analysis due to the large number of genes, but the limited number of protein biomarkers abstracted from the clustering analysis lends itself well to the genetic algorithm approach. Iterative analysis of algorithms (fitness tests) on a training set results in convergence to optimal predictive values which can then be tested on a validation set. Thus, in order to optimally select patient subsets with the best prognosis by a multiplex assay, we divided our entire cohort into two randomly assigned, training and validation sets with equal baseline survival.

Genetic algorithms are a mathematical approach to discover that replicates evolutionary processes. The genetic algorithm approach was invented in the late 1960's by John Holland. Like evolution, algorithms are tested for fitness, when fitness is described as a solution to a complex problem. Genetic algorithms borrow nomenclature from biology where each algorithm is a gene and genes are strung together into chromosomes. The solution is defined by the algorithms in the chromosome. The basic principles of genetic algorithms are that 1) they are defined by chromosomes, 2) the chromosomes are selected according to fitness and 3) they evolve either by mutation or cross-over to produce new generations of chromosomes.

This method is particularly well suited to analysis of multiplexed protein expression in tissue. We utilized a genetic algorithm to define a series of risk groups for disease specific death at 5 years. The genetic algorithm was utilized to develop a multiplex marker assay for separation of prognostic groups based on expression of ER, GATA3 and NAT1 A training set (n=223) and validation set (n=223) were randomly assigned in order to generate two groupings with equivalent baseline survival curves for all patients with values for all three markers and both censor and follow-up information. Data for each marker was normalized to control for differences in exposure time based on linear regression of redundant cases. One hundred random chromosomes were generated, each containing three genes (one each for ER, GATA3 and NAT1). Each gene represents an equation that is a true/false statement, such that the marker value is either greater than (>) or less than or equal to (<=) a randomly selected cutpoint. A chromosome thereby consists of three if/then statements that can be calculated as true or false. Each tumor is then assessed one point for each criteria that it meets when true, or no points when false. Tumors can thereby receive a 0, 1, 2 or a 3 as their cumulative score. These scores are referred to as A (3 points), B (2 points), C (1 point) and D (zero points). The predictive value of a chromosome was determined by assessing the risk ratio of the D group (high risk) compared to the A group (low risk). Genes were only evaluated if they resulted in at least 10% of the training set being assigned into the D group and at least 10% assigned into the A group in order to prevent finding groups of less than 20 patients each in the low and high risk groups. Chromosomes were subjected to multiple rounds of mutation (i.e. randomly changing the cutpoint value) or crossovers (e.g. replacing a marker cutpoint value with one from another intermediate risk 2 (score=2, Group B), or low risk (score=3, Group A). The chromosome converged after over 10,000 generations (iterations) with the best fit (highest relative risk between good and poor prognosis groups) was selected for validation: ER>4.6, GATA3>33.8, NAT1>18.6.

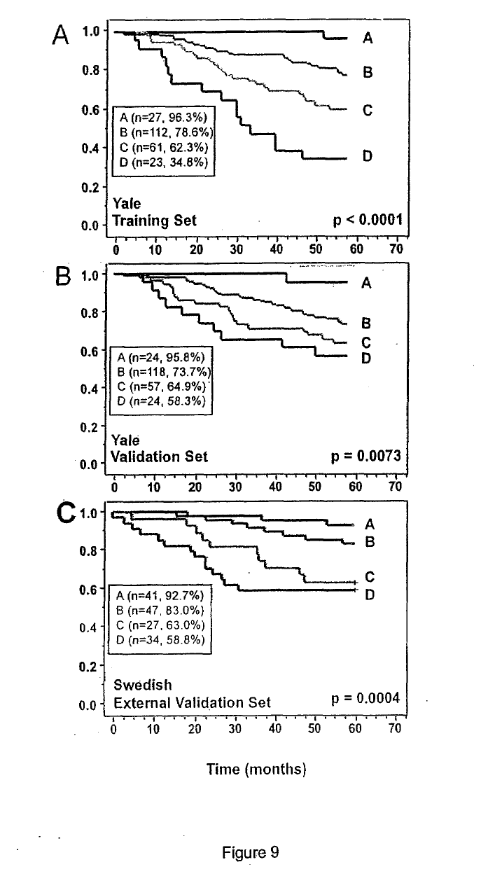

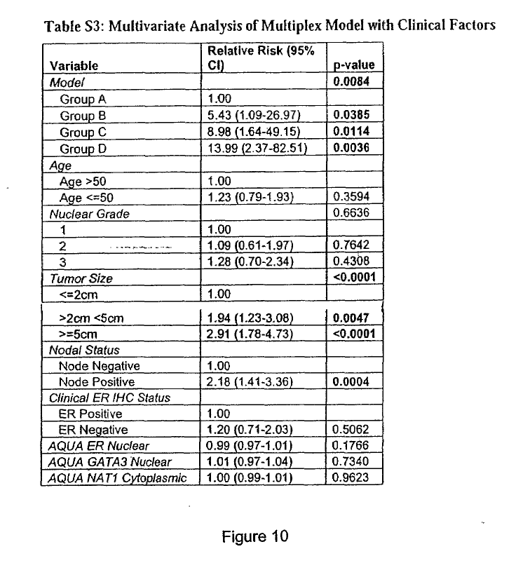

The application of the chromosome to the training set showed 27 patients were assigned to the good prognosis (low risk), with 1 event resulting in 96.3% survival at 5 years, and 23 patients assigned to the poor prognosis group (high risk) with 8 events (34.8% survival at 5 years). We were also able to assign patients into two intermediate risk groups, with 78.6% and 62.3% 5 year disease specific survival. The Kaplan Meier survival curves of the four groups for the training set are shown in FIG. 9A (log rank p<0.0001). We then applied this algorithm to the validation set, which resulted in the Kaplan Meier survival curves shown in FIG. 9B (log rank p=0.0073). In the validation set, the 24 patients in the low risk group had only 1 event (95.8% 5 year survival), and the 24 patients in the high risk group had 58.3% 5 year survival, showing again that our model was able to stratify patients into different prognostic groupings on the basis of a prospectively assignable score using only three protein expression measurements; this grouping remains independent in a multivariate Cox model including age at diagnosis, nuclear grade, tumor size, nodal status, clinical IHC ER status, and the individual AQUA scores for ER, GATA3 and NAT1 (FIG. 10).

For external validation of these findings, we repeated this assay on a cohort of patients from a randomized clinical trial of Tamoxifen conducted in Sweden from 1986 to 1991. A tissue microarray from these cases were analyzed for ER, GATA3 and NAT1, normalized for exposure times to the Yale cohort and then grouped as defined by the genetic algorithm above. The data from 149 patients from the control arm of this study (most similar to the patients in the Yale cohort) show a 95% five year survival in Group A compared to 58% five year survival in the high risk (Group D).

This study extends the multiplexing concept to protein expression analysis by quantitative extension of the familiar immunohistochemistry platform. This technology has the potential to easily translate into a valuable clinical test. It could be used to select a subset of breast cancer patients with a subclass of tumor that is highly unlikely to recur and thus the patients would require no therapy beyond surgical resection.

### REFERENCES

Incorporated by reference in their entirety are any polynucleotide and polypeptide sequences which reference an accession number correlating to an entry in the public database of the National Center for Biotechnology Information (NCBI) on the world wide web at ncbi.nlm.nih.gov. The contents of all cited references including literature references, issued patents, published or non published patent applications as cited throughout this application, including those references listed below, are hereby expressly incorporated by reference.
van't Veer, L. J., Paik, S. & Hayes, D. F. J. Clin Oncol 23, 1631-5 (2005); Paik, S. et al. N. Engl. J. Med 351, 2817-26 (2004); Ma, X. J. et al. Cancer Cell 5, 607-16 (2004); Foulkes, W. D. et al. Cancer Res 64, 830-5 (2004); Nielsen, T. O. et al. Clin Cancer Res 10, 5367-74 (2004); Kononen, J. et al. Nat Med 4, 844-7 (1998); Dolled-Filhart, M. & Rimm, D. L. in Cancer: Principles and Practice of Oncology (eds. DeVita, V. T., Jr., Hellman, S. & Rosenberg, S. A.) 26-34 (LWW Oncology, 2004); Camp, R. L., Chung, G. G. & Rimm, D. L. Nat Med 8, 1323-7 (2002); Dolled-Filhart, M. et al. Quantitative In Situ Analysis (AQUA) of Beta-Catenin Expression in Breast Cancer Shows Decreased Expression is Associated with Poor Outcome. (in submission) (2005); McCabe. T. Dolled-Filhart, M., Camp, R. L. & Rimm, D. L. Quantitative Analysis of Protein Expression shows that Changes in Antibody Concentration in Immunohistochemical Studies can Dramatically Affect Prognostic Significance, JNCI (in press) (2005); Perou, et al. Nature 406, 747-53 (2000); Sorlie, T. et al. Proc Natl Acad Sci USA 100, 8418-23 (2003); Malzahn, K., Mitze, M., Thoenes, M. & Moll, R. Virchows Arch 433, 119-29 (1998); Mitchell, M. Genetic Algorithms (MIT press, Cambridge, Mass., 1998); van't Veer, L. J. et al. Nature 415, 530-6 (2002); van de Vijver, M. J. et al. N. Engl. J. Med. 347, 1999-2009 (2002); Camp, R. L., Dolled-Filhart, M., King, B. L. & Rimm, D. L. Cancer Res 63, 1445-8 (2003); Camp, R. L., Rimm, E. B. & Rimm, D. L. Cancer 86, 2259-65 (1999); Chung, G. G. et al. Cancer 100, 2084-92 (2004); Kluger, H. M. et al. Clin Cancer Res 10, 173-7 (2004); Tolgay Ocal, I., Dolled-Filhart, M., D'Aquila, T. G., Camp, R. L. & Rimm, D. L. Cancer 97, 1841-8 (2003); Dolled-Filhart, M., Camp, R. L., Kowalski, D. P., Smith, B. L. & Rimm, D. L. Clin Cancer Res 9, 594-600 (2003); Cheadle, C., Vawter, M. P., Freed, W. J. & Becker, K. G. J. Mol. Diagn. 5, 73-81 (2003); Rubin, M. A. et al. Am. J. Pathol. 164, 831-40 (2004); Eisen, M. B., Spellman, P. T., Brown, P. O. & Botstein, D. Proc Natl Acad Sci USA 95, 14863-8 (1998); He, M., Burghardt, T. P. & Vockley, J. J. Biol. Chem. 278, 37974-86 (2003); Sorlie, T. et al. Proc Natl Acad Sci USA 98, 10869-74 (2001); Sotiriou, C. et al. Proc Natl Acad Sci USA 100, 10393-8 (2003); van't Veer, L. J. et al. Nature 415, 530-6 (2002); Thompson, D. A. & Weigel, R. J. Biochem Biophys Res Commun 251, 111-6 (1998); Cicatiello, L. et al. J. Mol. Endocrinol. 32, 719-75 (2004); Jiang, Y. et al. Discovery of differentially expressed genes in human breast cancer using subtracted cDNA libraries and cDNA microarrays. Oncogene 21, 2270-82 (2002); Mackay, A. et al. Oncogene 22, 2680-8 (2003); Beardsley, D. I. et al. Exp Cell Res 290, 402-13 (2003); Clark, J. et al. Genes Chromosomes Cancer 34, 104-14 (2002); Bertucci, F. et al. Hum Mol Genet 11, 863-72 (2002); West, M. et al. Proc Natl Acad Sci USA 98, 11462-7 (2001); Watson, P. H. et al. Br J Cancer 88, 1065-70 (2003); Bouras, T. et al. Cancer Res 62, 1289-95 (2002); Charpentier, A. H. et al. Cancer Res 60, 5977-83 (2000); Iwao, et al. Hum Mol Genet 11:199-206 (2002); Pusztai, et al. Clin Cancer Res 9, 2406-15 (2003); Zajchowski, D. A. et al Cancer Res 61, 5168-78 (2001); Gruvberger, S. et al. Cancer Res 61, 5 979-84 (2001); Oh, J. J., Grosshans, D. R., Wong, S. G. & Slamon, D. J. Nucleic Acids Res 27, 4008-17 (1999); Lin, C. Y. et al. Genome Biol 5, R66 (2004); Ahr, A. et al. J. Pathol 195, 312-20 (2001); Hoch, R. V., Thompson, D. A., Baker, R. J. & Weigel, R. J. International Journal of Cancer 84, 122-8 (1999); Rhee, M. S., Lindau-Shepard, B., Chave, K. J., Galivan, J. & Ryan, T. J. Mol. Pharmacol 53, 1040-6 (1998); Nacht, M. et al. Cancer Res 59, 5464-70 (1999); Martin, K. J. et al. Cancer Res 60, 2232-8 (2000); Hayashi, S. Biomed Pharmacother 58, 1-9 (2004); Inoue, A. et al. J. Mol. Endocrinol 29, 175-92 (2002); Wilson, K. S., Roberts, H., Leek, R., Harris, A. L. & Geradts, J. Am. J Pathol 161, 1171-85 (2002); Bosma, A. J. et al. Clin Cancer Res 8, 1871-7 (2002); Stanley, L. A. et al. J Histochem Cytochem 44, 1059-67 (1996); Itoh, T. et al. Mol Cancer Res 3, 203-18 (2005); Dunaway, G. A., Kasten, T. P., Sebo, T. & Trapp, R. Biochem J 251, 677-83 (1988); Stemmer-Rachamimov, et al. Am J Pathol 158, 57-62 (2001); Suemori, S., et al. Proc Natl Acad Sci USA 88, 11017-21 (1991); Porter, D. et al. Mol Cancer Res 1, 362-75 (2003); Camp, R. L., Dolled-Filhart, M., King, B. L. & Rimm, D. L. Cancer Res 63, 1445-8 (2003); Chung, G. G. et al. Cancer 100, 2084-92 (2004); Dolled-Filhart, M., Camp, R. L., Kowalski, D. P., Smith, B. L. & Rimm, D. L. Clin Cancer Res 9, 594-600 (2003); Kang, J. Y. et al. Cancer Res 63, 1101-5 (2003); Kluger, H. M. et al. Clin Cancer Res 10, 173-7 (2004); Tolgay Ocal, I., Dolled-Filhart, M., D'Aquila, T. G., Camp, R. L. & Rimm, D. L. Cancer 97, 1841-8 (2003); Ryden, L. et al. Eur J Cancer 41, 256-64 (2005); Pandolfi, P. P. et al. Nat Genet 11, 40-4 (1995); Ginestier, C. et al. Am J Pathol 161, 1223-33 (2002); van de Rijn, M. et al. Am J Pathol 161, 1991-6 (2002); Bertucci, F. et al. Hum Mol Genet 9, 2981-91 (2000); Schaner, M. E. et al. Mol Biol Cell 14. 4376-86 (2003); Jacquemier, et al J Cancer Res 65, 767-79 (2005); Usary, et al. Oncogene 23, 7669-78 (2004); Zheng, et al. J Natl Cancer Inst 90, 1724-9 (1998); Lee, K. M. et al. Cancer Lett 196, 179-86 (2003); Adam, P. J. et al. Mol Cancer Res 1, 826-35 (2003); Becker, M. et al. Mol Cancer Ther 4, 151-68 (2005); Parikh, P., Palazzo, J. P., Rose, L. J., Daskalakis, C. & Weigel, R. J. J Am Coll Surg 200, 705-10 (2005); Lee, J. H., Chung, J. G., Lai, J. M., Levy, G. N. & Weber, W. W. Cancer Lett 111, 39-50 (1997); Bieche, I., Girault, I., Urbain, E., Tozlu, S. & Lidereau, R. Breast Cancer Res 6, R252-63 (2004).

### EQUIVALENTS

The present invention provides, among other things, methods and compositions for diagnosing, prognosing and treating breast cancer. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

1. A method of determining whether a subject having breast cancer is likely to respond to a breast cancer therapy, comprising determining the ratio of HER2 expression to ER expression in a cell of said subject, wherein the determined ratio indicates whether the subject is likely to respond to the breast cancer therapy.

2. (canceled)

3. A method of selecting a breast cancer therapy for a subject, comprising determining the ratio of HER2 expression to ER expression in a cell of said subject, wherein the determined ratio indicates the appropriate therapy for the subject.

4. The method of claim 1, which further comprises determining the expression level of epidermal growth factor receptor (EGFR).

5. The method of claim 1, which further comprises determining the expression level of a member selected from the group consisting of HER1, HER2, HER3, HER4, PR, GATA3 and NAT1.

6. (canceled)

7. A method of diagnosing or prognosing breast cancer in a subject, comprising determining the ratio of HER2 expression to ER expression in a cell of said subject, wherein the determined ratio indicates the presence or stage of breast cancer in a subject and allows a prediction of the subject's chance of survival.

8. (canceled)

9. (canceled)

10. (canceled)

11. (canceled)

12. (canceled)

13. (canceled)

14. (canceled)

15. (canceled)

16. The method of claims 1, wherein the cell comprises a tissue sample.

17. The method of claim 16, wherein the tissue sample is present on a microarray.

18. (canceled)

19. A kit comprising reagents for detecting HER2 and ER.

20. (canceled)

21. A method of determining whether a subject having breast cancer is likely to respond to a breast cancer therapy, comprising quantifying the expression level in an appropriate cell of all of ER, GATA3, NAT1 and/or quantifying the expression level of at least two of EGFR, HER1, HER2, HER3, HER4, ER and PR, in a cell of said subject, wherein the determined ratio and/or quantified expression level indicates whether the subject is likely to respond to the breast cancer therapy.

22. (canceled)

23. A method of selecting a breast cancer therapy for a subject, comprising quantifying the expression level of all of ER, GATA3, NAT1 and/or quantifying the expression level of at least two of EGFR, HER1, HER2, HER3, HER4, ER and PR, in a cell of said subject, wherein the determined ratio or expression level indicates the appropriate therapy for the subject.

24. (canceled)

25. The method of claim 22, wherein the determined ratio indicates that the subject is likely to respond to HER2-based therapy but not hormone therapy.

26. The method of claim 22, wherein the determined ratio indicates that the subject is likely to respond to hormone therapy but not HER2-based therapy.

27. A method of diagnosing or prognosing breast cancer in a subject, comprising quantifying the expression level of all of ER, GATA3, NAT1 and/or quantifying the expression level of at least two of EGFR, HER1, HER2, HER3, HER4, ER and PR, in a cell of said subject, wherein the determined ratio or expression level indicates the presence or stage of breast cancer in a subject and allows a prediction of the subject's chance of survival.

28. (canceled)

29. (canceled)

30. (canceled)

31. The method of claim 27, wherein the level of expression is quantified by determining the AQUA™ score of ER, GATA3, NAT1, EGFR, HER1, HER2, HER3, HER4, 2C4, ER and/or PR.

32. The method of claim 31, wherein the AQUA™ score of ER, GATA3, NAT1, EGFR, HER1, HER2, HER3, HER4, 2C4, ER and/or PR is determined using the AQUA™ automated pathology system.

33. (canceled)

34. (canceled)

35. The method of claim 27, wherein the level of expression is quantified using a microarray.

36. The method of claim 35, wherein said tissue sample is present on a microarray.

37. The method of claim 28, wherein determining the level of expression comprises determining the location of ER, GATA3, NAT1, EGFR, HER1, HER2, HER3, HER4, ER and/or PR expression in the cell.

38. A kit comprising reagents for detecting ER, GATA3, NAT1, EGFR, HER1, HER2, HER3, HER4, 2C4, ER and/or PR.

39. (canceled)

40. (canceled)
